(19) United States
(12) Plant Patent Application Publication
(10) Pub. No.: US 2003/0196234 P1
(43) Pub. Date: Oct. 16, 2003

(54) KIWI PLANT NAMED 'HORTGEM WHA'

(76) Inventors: Mark A. McNeilage, Auckland (NZ); Ron A. Beatson, Motueka (NZ); Elspeth A. MacRae, Auckland (NZ)

Correspondence Address: GREENLEE WINNER AND SULLIVAN P C, 5370 MANHATTAN CIRCLE, SUITE 201, BOULDER, CO, 80303, US

(21) Appl. No.: 10/119,372

(22) Filed: Apr. 8, 2002

(51) Int. Cl. A01H 5/00

(52) U.S. Cl. PLT/156

(57) ABSTRACT

A new and distinct kiwi plant of the species Actinidia arguta (Sieb & Zucc.) Planch. ex Miq. var. arguta Li is described. The variety results from a controlled pollination using a female A. arguta selection AA05_01 of unknown parentage and a male A. arguta selection AA13_01 of unknown parentage. Both named parents (AA05_01 and AA13_01) are unpatented. The new variety is distinguished by its green hairless, edible skin, small fruit size, oblong shape and sweet aromatic taste.

### BACKGROUND TO THE INVENTION

Kiwi plants in cultivation are mainly varieties of A. deliciosa, particularly ‘Hayward’ although some A. chinensis and A. arguta varieties are grown. A. deliciosa and A. chinensis are closely related, whereas A. arguta is classified in a separate section of the genus. A. deliciosa and A. chinensis varieties have large fruit (˜100 g) with hair on the skin. The main varieties in New Zealand are ‘Hayward’ (A. deliciosa) and ‘Hort16A’ (A. chinensis). Fruit are usually cut and eaten with a spoon. A arguta has small fruit (˜10 g) with no hair on the skin. The skin is edible so these fruit can be eaten whole, like a grape.

All Actinidia species are dioecious, so female varieties have to be interplanted with male pollinizers to ensure fruit production.

A. arguta vines are deciduous and tend to grow vigorously in spring and summer when rapidly-growing shoots can intertwine and tangle if not managed. Vines do best in a mild temperate climate without late spring or early autumn frosts. They produce consistent heavy crops when grown in well-drained fertile soils and given regular irrigation in dry spells.

A. arguta flowers in spring (late October-early December) in New Zealand. Harvest of A. arguta fruit may occur between early February and late March in New Zealand depending on the selection and location of plantings. Compared to A. deliciosa and A. chinensis, A. arguta fruit require more careful handling during harvest and post-harvest procedures.

### SUMMARY OF THE INVENTION

The present invention is a new and distinctive kiwi plant having a small, generally oblong shaped fruit with green hairless edible skin. This new variety is designated ‘Hortgem Wha’ and is derived from a controlled pollination of AA05_{—}01, a female A. arguta selection of unknown parentage (unpatented), with AA13_{—}01, a male A. arguta selection of unknown parentage (unpatented).

The female parent arose from a seed family collected in the district Aomori, North Honshu, Japan and was introduced to New Zealand in 1977. The male parent was introduced as scionwood from the Royal Botanic Gardens, Edinburgh, Scotland to New Zealand in 1982. The provenance of both is unknown.

This new variety was created during the course of a planned plant-breeding program, which was initiated during 1987 at HortResearch in Auckland, New Zealand. The controlled cross was made in November 1987. Seeds were sown in autumn (March) 1988 and 102 seedlings from this cross were planted out in the field at HortResearch Kumeu Research Orchard in spring (October) 1988. The seedlings first fruited in February-March 1991. Twenty promising female seedlings were clonally propagated into a two-site replicated trial in 1995 and ‘Hortgem Wha’ (breeding code K2E5) was selected after storage and sensory evaluation in 1998.

The new variety can be asexually reproduced as cuttings or by grafting or budding on to seedling or cutting-grown rootstocks of A. arguta. Trial plantings as cuttings established in 1995 at Te Puke and Nelson Research Centres and on seedling rootstocks established in 1998 at these sites have shown that the unique combination of characters come true to form and are established and transmitted through succeeding asexual propagations.

### BRIEF DESCRIPTION OF THE ILLUSTRATIONS

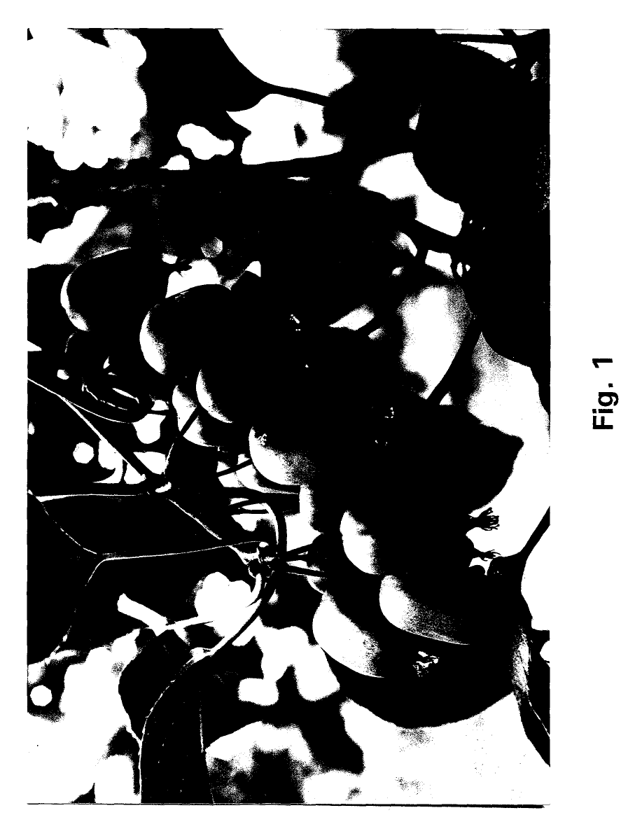

FIG. 1 shows typical fruit of the variety ‘Hortgem Wha’ in the orchard

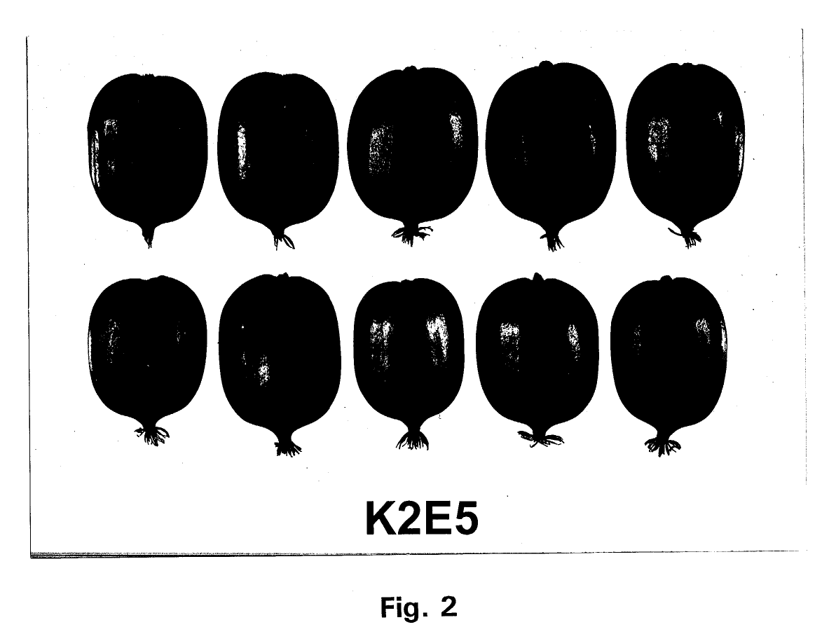

FIG. 2 shows typical fruit of the variety ‘Hortgem Wha’ in the studio

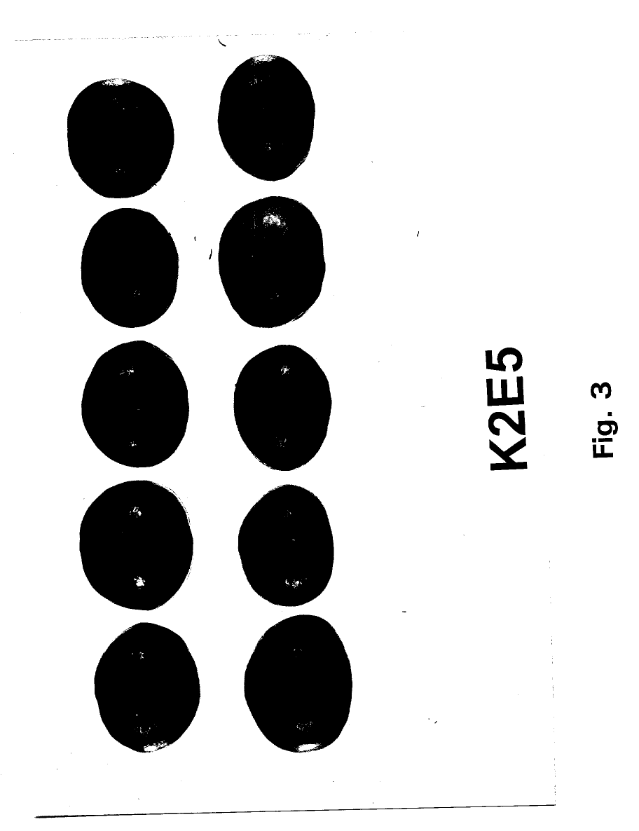

FIG. 3 shows a stem end view ‘Hortgem Wha’ fruit

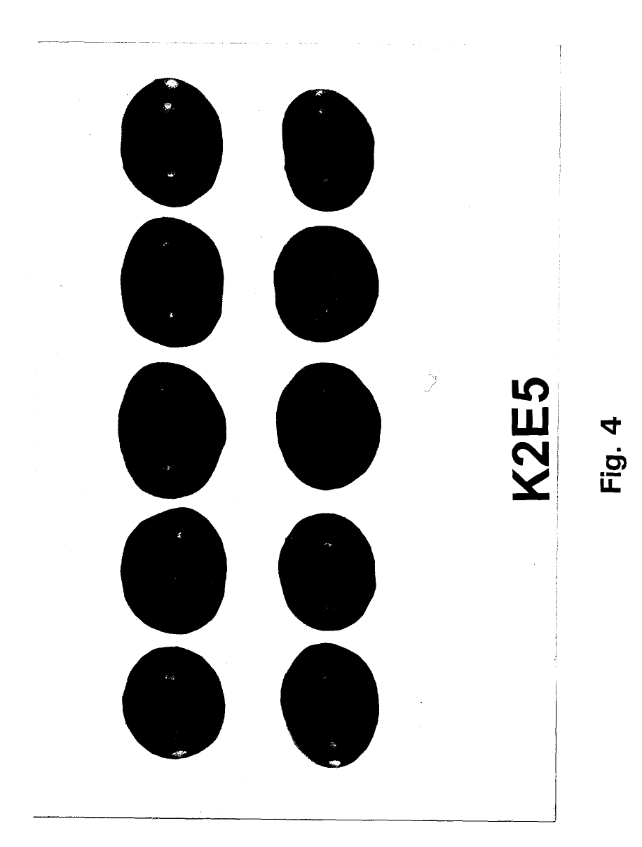

FIG. 4 shows a stylar end view of “Hortgem Wha” fruit

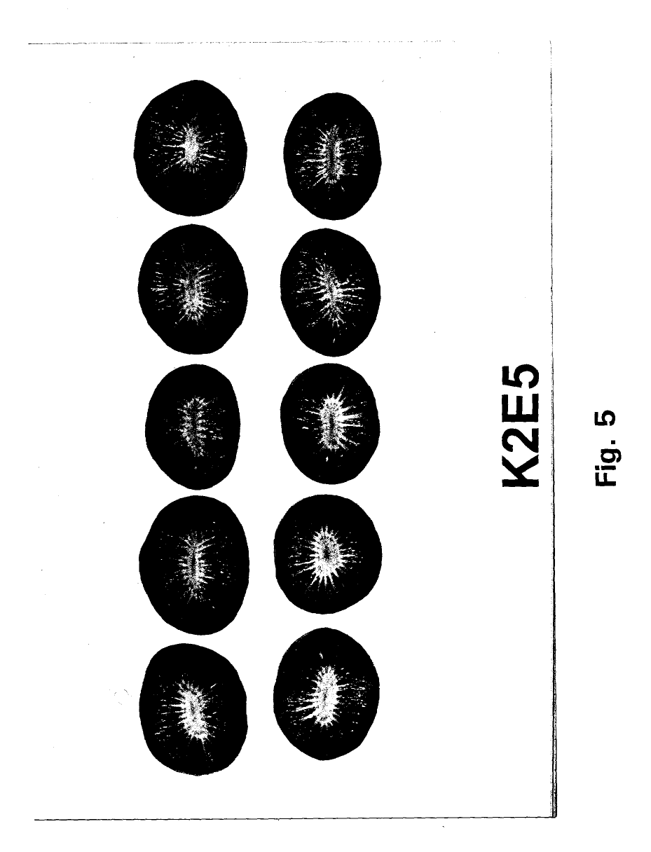

FIG. 5 shows ‘Hortgem Wha’ fruit in cross-section

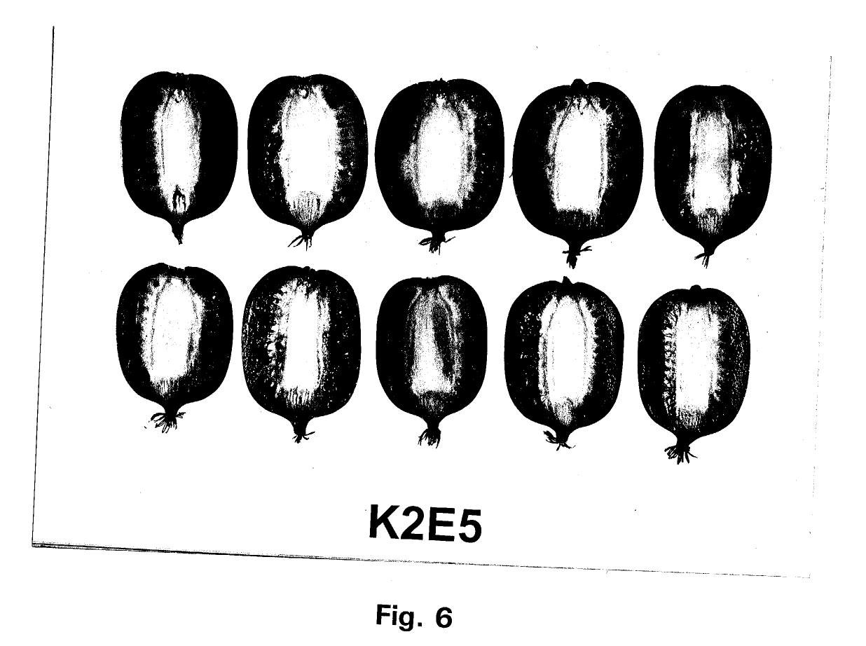

FIG. 6 shows ‘Hortgem Wha’ fruit in longitudinal-section

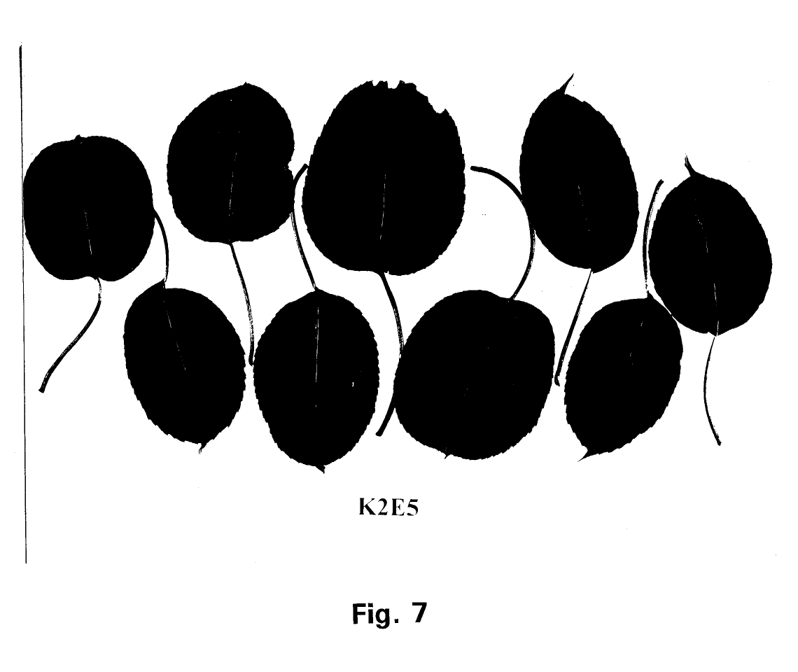

FIG. 7 shows leaves of the variety ‘Hortgem Wha’

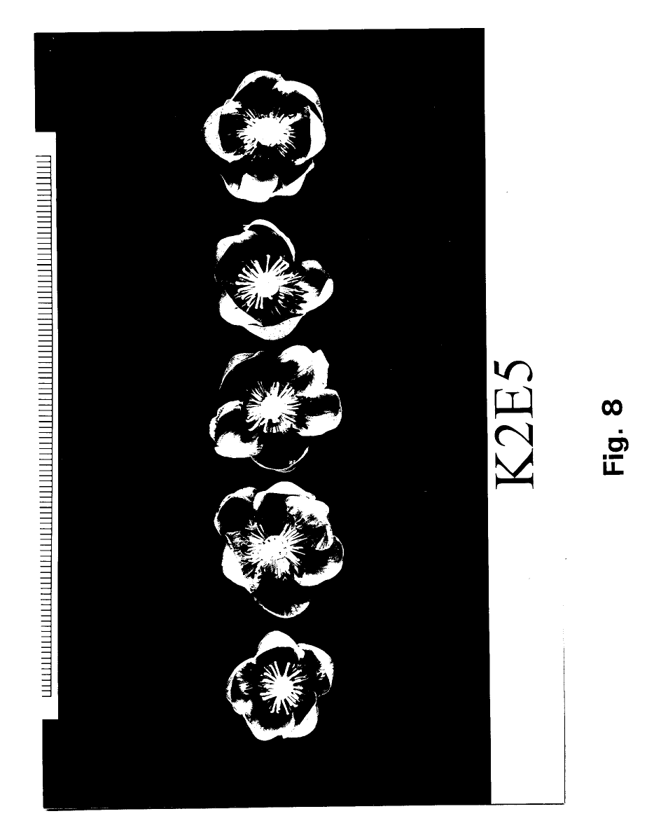

FIG. 8 shows flowers of the variety “Hortgem Wha’

Photographs of fruit were taken after the normal harvest date and are depicted in colors as nearly true as is reasonably possible to make the same in a color illustration of this character. Fruit skin color may vary depending upon extent of exposure to direct sunlight.

### COMPARISON TO CLOSEST VARIETY

The distinctive characteristics of this new kiwi variety, described in detail below, were observed in 2001 at Te Puke, New Zealand. The plants observed were established from cuttings and were 6 years old at the time. ‘Hortgem Wha’ is a new variety of A. arguta distinctive in its own right. Comparison with another similar variety ‘Hortgem Tahi’ (application Ser. No. 09/780,809) showed that ‘Hortgem Toru’ may be distinguished as follows:

| | | Expression | Expression of the |
|---|---|---|---|
| | | of the characteristics | characteristic for |
| | | for the similar | the candidate |
| Similar variety | Characteristic | variety | variety |
| Hortgem Tahi | Fruit shape | spheroid | oblong |
| Hortgem Tahi | Start of harvest | late Feb | late Feb-early |
| | | | March |
| Hortgem Tahi | Storage life of | ˜10-12 weeks | ˜6-8 weeks |
| | fruit at 0° C. | | |
| | in air storage | | |

### BOTANICAL DESCRIPTION OF THE PLANT

The new variety ‘Hortgem Wha’ is pistallate, with morphologically perfect but functionally imperfect flowers, i.e. the flowers produce only sterile pollen and thus require a pollinizer for fruit production. Characteristics of the new variety include a short period (˜3 months) between flowering and harvest, early harvest, small oblong fruit with green hairless edible skin, green flesh and a sweet aromatic flavour.

TABLE OF CHARACTERISTICS
Horticultural terminology is used in accordance with revised UPOV guide-
lines for kiwi. All dimensions in millimeters, weights in grams (unless
otherwise stated). Where a color reference is given these refer to the RHS
Colour Chart, the Royal Horticultural Society, London. 3^{rd} Edition, 1995.

| | |
|---|---|
| PLANT | |
| (Measurements from samples of 20, | |
| unless stated) | |
| Plant: sex expression | Female |
| Plant: vigour | Strong |
| Young shoot: hairs | Present |
| Young shoot: density of hairs | Sparse |
| Young shoot: type of hairs | Tomentose |
| Young shoot: anthocyanin coloration | Strong |
| of growing tip | |
| Young shoot: anthocyanin coloration | Absent or very weak |
| of leaf axil | |
| STEM | |
| Stem: coloration of leaf axil | Absent or very weak |
| Stem: diameter | Thin; mean 9.1 (range 8.5-10.0) |
| Stem: dormant bud diameter | Small, 1.2 mm (range 0.8-1.5) |
| Stem: color on upper side of shoot | Greyed-Orange 186B |
| Stem: character of bark | Smooth |
| Stem: hairs | Absent |
| Stem: conspicuousness of lenticels | Conspicuous |
| Stem: number of lenticels | Many |
| Stem: color of lenticels | Light red brown |
| Stem: size of bud support | 4.8 (range 3.8-5.7) |
| Stem: visibility of bud (dormant | Visible |
| canes) | |
| Stem: number of hairs visible on bud | Absent |
| (dormant canes) | |
| Stem: leaf scar | Deep |
| STEM (Mature) | |
| Leaf: general shape of blade | Ovate/broad ovate |
| Leaf: length | 115.4 mm (101-131) |
| Leaf: width | 68.4 mm (57-84) |
| Leaf: petiole length | 53.5 mm (43-73) |
| Leaf: shape of tip of blade | Caudate |
| Leaf: shape of base of blade | Rounded |
| Leaf: arrangement of leaf bases | Far apart |
| Leaf: puckering/blistering on upper | Absent or very weak |
| side of blade | |
| Leaf: margin | Ciliate |
| Leaf: green color of upper side of | Medium |
| blade | |
| Leaf: glossiness of upper side of | Medium |
| blade | |
| Leaf: color of lower side of blade | Light green |
| Leaf: glaucosity (lower side of blade) | Absent |
| Leaf: hairs on petiole | Absent or very weak |
| Leaf: density of hairs on petiole | Absent or very sparsek |
| Leaf: anthocyanin coloration on | Medium |
| upper side of petiole | |
| FLOWER | |
| Influorescence: predominant number | Three |
| of flowers: | |
| Pedicel: length | Short 28.1 mm (range 20.6-33.7) |
| Pedicel: hairs | Sparse |
| Pedicel: length of hairs | Very short |
| Flower: number of sepais | 5.8 (range 5-7) |
| Flower: color of sepais | Reddish brown |
| Flower: diameter (terminal or king | Small 28.2 (range 26.3-30.6) |
| flower when fully open) | |
| Flower: petal length | 15.1 (range 13.3-17.1) |
| Flower: petal width | 10.3 (range 8.7-12.4) |
| Flower: petal length/width ratio | 1.5 (range 1.12-1.84) |
| Flower: mean number of petals per | 6.1 (range 5-7) |
| flower | |
| Flower: number of flowers with more | 6 out of 20 (4 with 5, 10 with 6) |
| than six petals | |
| Flower: arrangement of petals | Overlapping |
| Flower: petal shoulder | Non-constrict |
| Flower: primary color of petals | Green white 157A |
| (when fully open) | |
| Flower: type of coloration of petals | Uniform over entire petal |
| Flower: number of stamens | 44.4 (range 36-49) |
| Flower: length of stamen filaments | 4.2 (range 3.5-4.7) |
| Flower: length of anthers | 2.4 (range 2.1-2.6) |
| Flower: filament color | Light green |
| Flower: anther color | Black |
| Flower: style number | 23.8 (range 21-28) |
| Flower: length of styles | 3.7 (range 3.0-4.2) |
| Flower: attitude of styles | Semi-erect |
| Flower: curvature of styles | Weak |
| Flower: length of ovary | 7.9 (range 7.2-8.5) |
| Flower: amount of hair on ovary | Absent |
| Flower: color of ovary | Yellow green 145A |
| FRUIT | |
| Fruit: overall size | 8.5 g (7.4-11.4) |
| Fruit: length | 32.0 mm (30-40) |
| Fruit: width (max) | 23.3 mm (21-26) |
| Fruit: width (min) | 19.7 mm (18-22) |
| Fruit: core diameter (max) | 8.6 mm (6-12) |
| Fruit: core diameter (min) | 4.2 mm (3-5) |
| Fruit locule number | 23.6 (19-27) |
| Fruit peduncle length | 27.9 mm (20-36) |
| Fruit: peduncle width | 1.7 mm (1.3-2.3) |
| Fruit: general shape | Oblong |
| Fruit: cross section ar median | Oblate |
| Fruit: general shape of stylar end | Slightly pointed protruding |
| Fruit: shape of shoulder on stalk end | Square |
| Fruit: skin color at harvest (fruit | Medium green |
| still hard) | |
| Fruit: skin color change during | Medium green |
| ripening | |
| Fruit: skin color at maturity for | Medium green |
| consumption | |
| Fruit: hairs | Absent |
| Fruit: core diamerter (at largest | Medium |
| diameter) | |
| Fruit: core shape (in cross | Transverse elliptic |
| section) | |
| Fruit: core woody spike | Absent |
| Fruit: outer pericarp color at | Green |
| maturity for consumption | |
| Fruit: inner pericarp col. (locules) | Green |
| at mat. for consumption | |
| Fruit: core color at maturity | Green white |
| Fruit: seed color at maturity (in flesh) | Dark red brown |
| Fruit: seed colour when dry | Red brown |
| EVENTS | |
| Time of vegetative budbreak | Early-mid September |
| Time of beginning of flowering | Early-mid November |
| Time of maturity for harvest (at nor- | Late February-early March |
| malized Brix level) | |
| Fruit: outer pericarp color at matur- | 139B/141B |
| ity for consumption | |
| Fruit: inner pericarp col. (locules) at | 139B/141B |
| mat. for consumtpion | |
| Fruit: core color at harvest | 154D/157C |
| Fruit: seed color (in flesh) | 172B/175C |
| Fruit: seed solour (dry seed) | 172B/175C |
| Fruit: skin color at maturioty | 141A/143A |
| Leaf: color upper side (in mature leaf | 136A/139A |
| after petal fall) | |
| Leaf: color of flower side (in mature | 138A/141A |
| leaf after petal fall) | |
| Flower: color of main body of petals | 157A |
| Flower: color of ovary | 145A |
| Plant stem: color on exposed side | 166B/176A |

### HORTICULTURAL CHARACTERISTICS

Details below relate to observations made on cutting-grown plants growing at HortResearch Te Puke Research Centre, New Zealand. These vines were 3 years old.

Cropping: young vines of ‘Hortgem Wha’ are precocious, beginning to bear in their second year and are expected to reach full capacity at about 7 years. The storage life of ‘Hortgem Wha’ fruit is 6-8 weeks at 0° C., if stored in unventilated containers in air storage.

Yield and fruit size: (Data from harvesting all fruit from 8 vines in early March 1999)

Mean fruit weight: 7.34 g Maximum: 7.85 g Minimum: 6.53 g

Mean fruit number: 845 Maximum: 1,498 Minimum: 297

Mean yield: 6.2 kg Maximum: 11.1 kg Minimum: 2.3 kg

It is anticipated mature, well-managed vines would yield approximately 5000 fruit per vine with a mean fruit weight of 10 g and mean yield per vine of 50 kg.

1. A new and distinct kiwi plant of the species A. arguta substantially as herein described and illustrated, characterized by small oblong fruit with green hairless edible skin and green flesh with a sweet aromatic flavor.
